(19) United States
(12) Patent Application Publication
(10) Pub. No.: US 2025/0066735 A1
(43) Pub. Date: Feb. 27, 2025

(54) PREPARATION METHOD OF MESENCHYMAL STEM CELL (MSC) AND APPLICATION THEREOF IN KNEE PRODUCT

(71) Applicant: GUANGDONG CELL BIOTECHNOLOGY CO. LTD, Dongguan (CN)

(72) Inventor: Xingxin WANG, Dongguan (CN)

(73) Assignee: GUANGDONG CELL BIOTECHNOLOGY CO. LTD, Dongguan (CN)

(21) Appl. No.: 18/948,773

(22) Filed: Nov. 15, 2024

(30) Foreign Application Priority Data
Jul. 18, 2024 (CN) 202410961942.X

(51) Int. Cl. C12N 5/0775

(52) U.S. Cl. CPC C12N 5/0663; C12N 2500/32; C12N 2500/35

(57) ABSTRACT

The present disclosure provides a preparation method of a mesenchymal stem cell (MSC) and an application thereof in a knee product. The additive composition including NOG and quercetin provided by the present disclosure includes components that are combined scientifically and reasonably and play a synergistic role, and has a stable efficacy.

### CROSS-REFERENCE TO RELATED APPLICATIONS

This application claims priority to Chinese Patent Application No. 202410961942.X with a filing date of Jul. 18, 2024. The content of the aforementioned application, including any intervening amendments thereto, is incorporated herein by reference.

### TECHNICAL FIELD

The present disclosure belongs to the field of stem cell technologies, and specifically relates to a preparation method of a mesenchymal stem cell (MSC) and an application thereof in a knee product.

### BACKGROUND

MSCs are a class of stem cells with self-renewal and multi-directional differentiation potential. MSCs also have low immunogenicity and an immunomodulatory effect. MSCs play an important role in medical research and clinical practice. MSCs can be isolated from tissues such as bone marrow, fat, umbilical cord blood, and amniotic fluid. MSCs derived from different tissues have different immunomodulatory functions. The use of some cytokines, growth factors, hormones, vitamins, antioxidants, anti-tumor drugs, etc. alone or in their combinations can induce the differentiation of MSCs into a specified cell lineage under in vitro cultivation conditions.

The main pathological feature of osteoarthritis is a degenerative disease of articular cartilage, and the most common disease is knee osteoarthritis. The long-term, heavy, and high-load exercise, the osteoarthritis in middle-aged and elderly people, etc. may cause damage to knee articular cartilage, which further develops into an articular cartilage lesion and evolves into knee osteoarthritis. Articular cartilage is a connective tissue with a special structure. There are no blood vessels and nervous systems in articular cartilage, and thus articular cartilage does not have a self-healing ability after injury. Currently, the corresponding treatments include medication, surgery, and autologous chondrocyte implantation. However, these treatments can only play a relieving role, and exhibit a weak cartilage regeneration effect. MSCs can achieve the regeneration of knee articular cartilage, which provides a new option for the treatment of cartilage injury in knee osteoarthritis.

However, the existing evidence shows that bone marrow-derived MSCs transplanted have a low survival rate in vivo, and lead to limited improvement effects for symptoms such as knee osteoarthritis. Therefore, it is necessary to develop a method for cultivating MSCs that can improve both a survival rate of MSCs and a symptom of knee osteoarthritis.

N-oxalylglycine (NOG) is a broad-spectrum 2-oxoglutarate oxygenase inhibitor with an inhibitory activity against prolyl hydroxylase domain (PHD). PHD can hydroxylate the hypoxia-inducible factor (HIF) to promote the degradation of HIF, thereby affecting the treatment of HIF-mediated diseases. After chondrocytes, osteoblasts, or the like recognize the low oxygen pressure at the bone injury site, HIF, as an upstream regulator of bone repair and regeneration, can activate the HIF pathway and increase the expression of HIF-la and downstream genes thereof, thereby stimulating the generation of cardiac blood vessels and the early transport and differentiation of osteoblasts. Therefore, NOG can inhibit an activity of PHD to further inhibit the degradation of HIF, thereby promoting the proliferation of osteoblasts.

### SUMMARY

In view of the technical status quo, the technical problem to be solved by the present disclosure is to overcome the problem that the existing MSCs have a low survival rate and lead to limited improvement effects for knee osteoarthritis after being transplanted, and to provide a preparation method of MSC and an application thereof in a knee product. The composition including NOG and quercetin provided by the present disclosure includes components that are combined scientifically and reasonably and play a synergistic role, and has a stable efficacy.

A first objective of the present disclosure is to provide a preparation method of MSC. Specifically, a composition including NOG and quercetin is used as an additive to improve a survival rate of bone marrow-derived MSCs after being transplanted.

A second objective of the present disclosure is to provide an application of the preparation method of MSC in preparation of a knee product.

A third objective of the present disclosure is to provide an application of a bone marrow-derived MSC prepared by the preparation method of MSC in a treatment of knee osteoarthritis.

The above objectives of the present disclosure are implemented through the following technical solutions:

A preparation method of an MSC is provided, including: cultivating a third-generation human bone marrow-derived MSC produced after passage using a modified cell culture medium to obtain a bone marrow-derived MSC for cell transplantation.

In the preparation method of the MSC, the modified cell culture medium is obtained by adding a composition of NOG and quercetin to a basal medium.

A concentration of the composition of NOG and quercetin in the modified cell culture medium is 0.5 μmol/mL to 2.0 μmol/mL, and a concentration of the quercetin in the modified cell culture medium is 0.1 μmol/mL to 5.0 μmol/mL.

A molar ratio of the NOG to the quercetin is 1:(0.05-20).

Optionally, the molar ratio of the NOG to the quercetin is 1:0.1.

Optionally, the concentration of the NOG is 1.0 μmol/mL and the concentration of the quercetin is 0.1 μmol/mL.

In the preparation method of the MSC, the third-generation human bone marrow-derived MSC is cultivated in the modified cell culture medium for 1 h to 24 h and optionally for 24 h.

A modified MSC culture medium is provided, including a basal medium, NOG, and quercetin; a concentration of the NOG in the modified MSC culture medium is 0.5 μmol/mL to 2.0 μmol/mL, a concentration of the quercetin in the modified MSC culture medium is 0.1 μmol/mL to 5.0 μmol/mL, and a molar ratio of the NOG to the quercetin is 1:(0.05-20).

Optionally, the molar ratio of the NOG to the quercetin is 1:0.1.

Optionally, the concentration of the NOG is 1 μmol/mL and the concentration of the quercetin is 0.1 μmol/mL.

The method of the present disclosure has the following beneficial effects:

(1) The present disclosure can promote the proliferation of bone marrow-derived MSCs and significantly improve a survival rate of the bone marrow-derived MSCs by scientifically setting a composition ratio of NOG to quercetin.

(2) Bone marrow-derived MSCs cultivated by the method of the present disclosure can improve the symptoms of knee osteoarthritis, and have a promising application prospect.

### BRIEF DESCRIPTION OF THE DRAWING

FIG. 1 shows the optimal action time of an additive composition.

### DETAILED DESCRIPTION OF THE EMBODIMENTS

The examples of the present disclosure are described in detail below. The examples provided by the present disclosure are illustrative, and are intended to explain the present disclosure rather than limit the present disclosure.

### Example 1

(1) Isolation of bone marrow-derived MSCs from a bone marrow: 20 mL to 40 mL of a human bone marrow was extracted and added to a sterile glass bottle with 3,000 U heparin, and the human bone marrow and the heparin were fully mixed for anticoagulation. Normal saline was added at the same amount as the human bone marrow to the sterile glass bottle, and mixing was conducted thoroughly to obtain diluted human bone marrow. The diluted human bone marrow was added slowly to a Ficoll liquid level with 1 part by volume of a lymphocyte separation solution (Ficoll separation solution) per 2 parts by volume of the diluted human bone marrow to obtain a mixed system. The mixed system was centrifuged at 2,000 r/min and 20° C. in a horizontal centrifuge for 25 min, and bone marrow-derived MSCs were collected from an interface, washed with normal saline 2 to 3 times, suspended with an appropriate amount of a culture medium, and counted.

(2) Primary cultivation of the bone marrow-derived MSCs: The bone marrow-derived MSCs obtained in the step (1) were inoculated at a density of 0.3×10⁶/mL in a basal medium, and the primary cultivation was conducted. The basal medium is Dulbecco's Modified Eagle Medium (DMEM, GIBCO company, America). During the primary cultivation, the medium was changed every 2 d to remove non-adherent cells and continuously purify the human bone marrow-derived MSCs. When a cell density reached 80%, the sub-cultivation was conducted. For an experimental group, an additive composition of NOG and quercetin was added to the basal medium to prepare an experimental medium.

The sub-cultivation was conducted until third-generation human bone marrow-derived MSCs were obtained. The third-generation human bone marrow-derived MSCs were further cultivated with the experimental medium for cell transplantation.

(3) A survival rate of the human bone marrow-derived MSCs was detected with Trypan blue. The human bone marrow-derived MSCs obtained in the step (2) (cells cultivated without NOG and quercetin were adopted as a control group) were transferred to a low-sugar DMEM including 100 mM hydrogen peroxide (H₂O₂), cultivated for 1 h, stained with Trypan blue, and observed under a microscope to count a cell survival rate. Test results were shown in Table 1 below.

TABLE 1
Cell survival rates under cultivation conditions of
different additive contents

| | | Cell |
|---|---|---|
| Group | Medium additive | survival rate |
| Group 1 | 0.5 μmol/mL of NOG + 0.1 μmol/mL of quercetin | 67.8 ± 1.5% |
| Group 2 | 1.0 μmol/mL of NOG + 0.1 μmol/mL of quercetin | 91.3 ± 0.5% |
| Group 3 | 2.0 μmol/mL of NOG + 0.1 μmol/mL of quercetin | 80.2 ± 1.1% |
| Group 4 | 1.0 μmol/mL of NOG + 3.0 μmol/mL of quercetin | 60.5 ± 0.7% |
| Group 5 | 1.0 μmol/mL of NOG + 5.0 μmol/mL of quercetin | 55.6 ± 2.5% |
| Group 6 | 2.0 μmol/mL of NOG + 3.0 μmol/mL of quercetin | 70.3 ± 0.3% |
| Group 7 | 2.0 μmol/mL of NOG + 5.0 μmol/mL of quercetin | 65.4 ± 1.3% |
| Group 8 | 0.5 μmol/mL of NOG + 3.0 μmol/mL of quercetin | 53.4 ± 0.4% |
| Group 9 | 0.5 μmol/mL of NOG + 5.0 μmol/mL of quercetin | 49.2 ± 0.6% |
| Control | 0 μmol/mL of NOG + 0 μmol/mL of quercetin | 47.8 ± 1.3% |
| group | | |

A composition of NOG and quercetin was added to the basal medium to prepare the experimental medium. A concentration of NOG in the experimental medium was 0.5 μmol/mL to 2.0 μmol/mL, a concentration of quercetin in the experimental medium was 0.1 μmol/mL to 5.0 μmol/mL, and a molar ratio of NOG to quercetin was 1:(0.05-20). The experimental medium can promote the proliferation of bone marrow-derived MSCs and significantly improve a survival rate of bone marrow-derived MSCs. A cell survival rate is not positively correlated with a single component in the composition. A composition with an NOG concentration of 1.0 μmol/mL and a quercetin concentration of 0.1 μmol/mL allowed the optimal effect, and thus cells treated with this composition were selected for follow-up experiments.

TABLE 2
Impacts of different additive components and different amounts
of each additive component on a cell survival rate

| | | Cell |
|---|---|---|
| Group | Medium additive | survival rate |
| Comparative | 0 μmol/mL of NOG + 0.1 | 50.2 ± 0.7% |
| Example 1 | μmol/mL of quercetin | |
| Comparative | 1.0 μmol/mL of NOG + 0 | 52.3 ± 0.8% |
| Example 2 | μmol/mL of quercetin | |
| Comparative | 1.0 μmol/mL of NOG + 0.1 | 70.2 ± 0.3% |
| Example 3 | μmol/mL of isoliquiritigenin | |
| Comparative | 1.0 μmol/mL of dimethyloxalylglycine | 81.9 ± 0.5% |
| | (DMOG) + 0.1 μmol/mL of | |
| Example 4 | quercetin | |

In order to prove a synergistic effect of components in the additive composition of the present disclosure, the present disclosure provided the comparative examples. Test results were shown in Table 2. The addition of 0.1 μmol/mL of quercetin alone, or the addition of 1.0 μmol/mL of NOG alone, or the use of isoliquiritigenin with a similar effect and efficacy to quercetin instead in combination with NOG all lead to an inferior cell survival rate-improving effect to the composition of NOG and quercetin in the present disclosure. The use of DMOG as a PHD inhibitor in combination with quercetin also has an inferior cell survival rate-improving effect to the composition of NOG and quercetin in the present disclosure.

The additive composition of NOG and quercetin in the present disclosure is not a simple combination. In group 3 and group 4 of Example 1, a content of a single component in the composition is increased, but a cell survival rate-improving effect is not increased. In the comparative examples, a component in the composition is substituted with another component at a same amount, but a cell survival rate-improving effect is not better than the composition with an NOG concentration of 1.0 μmol/mL and a quercetin concentration of 0.1 μmol/mL in the present disclosure. In summary, in the composition with an NOG concentration of 1.0 μmol/mL and a quercetin concentration of 0.1 μmol/mL in the present disclosure, NOG and quercetin play a synergistic role.

### Example 2 Investigation of the Optimal Action Time of the Additive Composition

The step (3) in Example 1 was repeated. Cells treated with a low-sugar DMEM that did not include the composition of NOG and quercetin and included only H₂O₂ were taken as a control group, and the cells were cultivated for 1 h, 12 h, and 24 h. Cells treated with a low-sugar DMEM that included the composition with an NOG concentration of 1.0 μmol/mL and a quercetin concentration of 0.1 μmol/mL were taken as an experimental group, and the cells were cultivated for 1 h, 12 h, and 24 h. A survival rate of the human bone marrow-derived MSCs was detected with Trypan blue. Specific experimental results were detailed in Table 3.

TABLE 3
Results of investigation of the optimal action time
of the additive composition

| | Treatment time | | |
|---|---|---|---|
| Group | 1 h | 12 h | 24 h |
| Control group | 47.8 ± 1.3% | 47.0 ± 0.9% | 46.9 ± 1.1% |
| Experimental group | 85.3 ± 0.5% | 86.5 ± 0.8% | 89.7 ± 0.9% |

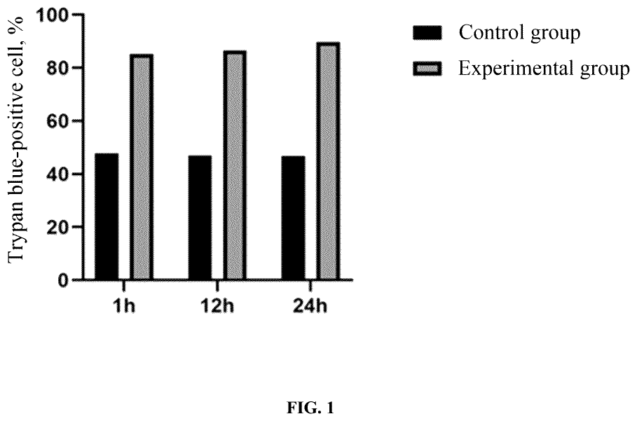

The results in Table 3 and FIG. 1 show that the treatment with the composition of NOG and quercetin for 24 h leads to the optimal effect for improving a survival rate of bone marrow-derived MSCs.

### Example 3 Construction of a Rat Knee Osteoarthritis Model

8 rats were randomly selected as a blank control group. Sodium iodoacetate with a mass concentration of 20 mg/mL was injected into knee joint cavities of rats at 0.1 mL/rat, and then the rats were randomly divided into a model group, a positive control group, and an experimental group with 8 rats in each group. Rats in the blank control group were allowed to eat and drink freely. Rats in the model group were administered with normal saline. Rats in the positive control group were injected with bone marrow-derived MSCs not treated by the composition of NOG and quercetin in the present disclosure. Rats in the experimental group were injected with bone marrow-derived MSCs treated by the composition of NOG and quercetin in the present disclosure.

For the experimental group, bone marrow-derived MSCs treated with the optimal additive composition of NOG (concentration: 1 mol/mL) and quercetin (concentration: 0.1 μmol/mL) for the optimal action time (24 h) screened in Examples 1 and 2 were suspended with normal saline to prepare a total of 50 μL of a bone marrow-derived MSC suspension in which a concentration of the bone marrow-derived MSCs was 2×10⁶ cells/mL.

On day 6 and day 12 of the experiment, changes in joint diameters of rats in each group were observed.

TABLE 4
Changes in joint diameters of rats in each group

| | Before | After administration | | |
|---|---|---|---|---|
| Group | molding | Day 0 | Day 6 | Day 12 |
| Blank | 0.434 ± 0.005 | 0.436 ± 0.003 | 0.448 ± 0.004 | 0.447 ± 0.003 |
| control | | | | |
| group | | | | |
| Model | 0.434 ± 0.004 | 0.491 ± 0.035 | 0.488 ± 0.023 | 0.490 ± 0.012 |
| group | | | | |
| Positive | 0.434 ± 0.004 | 0.498 ± 0.034 | 0.476 ± 0.030 | 0.463 ± 0.005 |
| control | | | | |
| group | | | | |
| Ex- | 0.434 ± 0.003 | 0.499 ± 0.032 | 0.458 ± 0.03 | 0.441 ± 0.006 |
| perimental | | | | |
| group | | | | |

On day 6 after administration, changes in joint diameters of rats in the experimental group and the positive control group compared with the model group are statistically significant, and a change of a joint diameter of rats in the blank control group compared with the model group is not statistically significant, indicating that the bone marrow-derived MSCs of the present disclosure have an improvement effect on a knee injury in rats. On day 12 after administration, joint diameters of rats in the experimental group and the positive control group significantly change compared with the model group, and an improvement effect of the experimental group is better than an improvement effect of the positive control group, indicating that the bone marrow-derived MSCs prepared with the optimal additive composition of NOG (concentration: 1.0 mol/mL) and quercetin (concentration: 0.1 μmol/mL) in the present disclosure has the optimal effect for improving a knee injury in rats.

Apparently, the above examples are merely intended to describe the present disclosure clearly, rather than to limit the implementations of the present disclosure. Those of ordinary skill in the art may make modifications or variations in other forms based on the above description. There are no need and no way to exhaust all of the implementations. Any modification, equivalent substitution, and improvement made based on the present disclosure should fall within the protection scope of the claims of the present disclosure.

What is claimed is:

1. A preparation method of a bone marrow-derived mesenchymal stem cell (MSC), comprising: cultivating a third-generation human bone marrow-derived MSC produced after passage using a modified cell culture medium, to obtain a bone marrow-derived MSC for cell transplantation,
wherein the modified cell culture medium is obtained by adding a composition of N-oxalylglycine (NOG) and quercetin to a basal medium; and a concentration of the NOG in the modified cell culture medium is 1.0 μmol/mL and a concentration of the quercetin in the modified cell culture medium is 0.1 μmol/mL.

2. A modified MSC culture medium, comprising a basal medium, NOG, and quercetin, wherein a concentration of the NOG in the modified MSC culture medium is 1 μmol/mL and a concentration of the quercetin in the modified MSC culture medium is 0.1 μmol/mL.
